(19) United States
(12) Patent Application Publication
(10) Pub. No.: US 2024/0152586 A1
(43) Pub. Date: May 9, 2024

(54) ECG-BASED BIOMETRIC AUTHENTICATION

(71) Applicant: AliveCor, Inc., Mountain View, CA (US)

(72) Inventor: Vish Chidambaram, Fremont, CA (US)

(21) Appl. No.: 17/983,148

(22) Filed: Nov. 8, 2022

(51) Int. Cl. A61B 5/316

(52) U.S. Cl. CPC A61B 5/316; A61B 5/346

(57) ABSTRACT

Embodiments of the present disclosure provide systems and methods for performing ECG-based authentication. A set of electrocardiogram (ECG) signals of a user may be received by a computing device from an ECG monitor. The computing device may be associated with a resource to which the computing device controls access. An ECG profile for the user may be determined based on the set of ECG signals. In response to receiving a request to access the resource, an authentication ECG signal may be requested to authenticate the user. The authentication ECG signal may be received from the ECG monitor and compared to the ECG profile of the user to determine whether the authentication ECG signal matches the ECG profile. In response to determining that the authentication ECG signal matches the ECG profile, the user may be authenticated and granted access to the resource.

### TECHNICAL FIELD

Aspects of the present disclosure relate to electrocardiogram (ECG) based authentication.

### BACKGROUND

An ECG monitoring device may comprise a set of electrodes for recording ECG waveforms (also referred to herein as “taking an ECG”) of the person's heart. The set of electrodes may be placed on the skin of the person in multiple locations and the electrical signal recorded between each electrode pair (ECG waveform) in the set of electrodes may be referred to as a lead. The ECG waveforms (each one corresponding to a lead of the ECG) recorded by the ECG monitoring device may comprise data corresponding to the electrical activity of the person's heart. Varying numbers of leads can be used to take an ECG, and different numbers and combinations of electrodes can be used to form the various leads. Example numbers of leads used for taking ECGs are 1, 2, 6, and 12 leads.

### BRIEF DESCRIPTION OF THE DRAWINGS

The described embodiments and the advantages thereof may best be understood by reference to the following description taken in conjunction with the accompanying drawings. These drawings in no way limit any changes in form and detail that may be made to the described embodiments by one skilled in the art without departing from the spirit and scope of the described embodiments.

FIG. 1A is a block diagram that illustrates an example system, in accordance with some embodiments of the present disclosure.

FIG. 1B illustrates a single dipole heart model with a 12 lead set represented on a hexaxial system, in accordance with some embodiments of the present disclosure.

FIG. 1C illustrates a single dipole heart model with a 12 lead set represented on a hexaxial system, in accordance with some embodiments of the present disclosure.

FIG. 2A is a block diagram that illustrates an example ECG monitor, in accordance with some embodiments of the present disclosure.

FIG. 2B is a block diagram that illustrates the hardware design of an example ECG monitor, in accordance with some embodiments of the present disclosure.

FIG. 3 is a block diagram that illustrates an ECG profile and authentication ECG signal comparison process, in accordance with some embodiments of the present disclosure.

FIG. 4A is a block diagram that illustrates another example system, in accordance with some embodiments of the present disclosure.

FIG. 4B is a block diagram that illustrates another example system, in accordance with some embodiments of the present disclosure.

FIG. 5 is a block diagram that illustrates a combined ECG monitor and fingerprint scanning device, in accordance with some embodiments of the present disclosure.

FIG. 6 is a flow diagram of a method of performing ECG-based authentication, in accordance with some embodiments of the present disclosure.

FIG. 7 is a block diagram of an example computing device that may perform one or more of the operations described herein, in accordance with some embodiments of the present disclosure.

### DETAILED DESCRIPTION

A typical heartbeat may include several variations of electrical potential, which may be classified into waves and complexes, including a P wave, a QRS complex, a T wave, and a U wave among others, as is known in the art. Stated differently, each ECG waveform may include a P wave, a QRS complex, a T wave, and a U wave among others, as is known in the art. The shape and duration of these waves may be related to various characteristics of the person's heart such as the size of the person's atrium (e.g., indicating atrial enlargement) and can be a first source of heartbeat characteristics unique to a person. The ECG waveforms may be analyzed (typically after standard filtering and “cleaning” of the signals) for various indicators that are useful in detecting cardiac events or status, such as cardiac arrhythmia detection and characterization. Such indicators may include ECG waveform amplitude and morphology (e.g., QRS complex amplitude and morphology), R wave-ST segment and T wave amplitude analysis, and heart rate variability (HRV), for example.

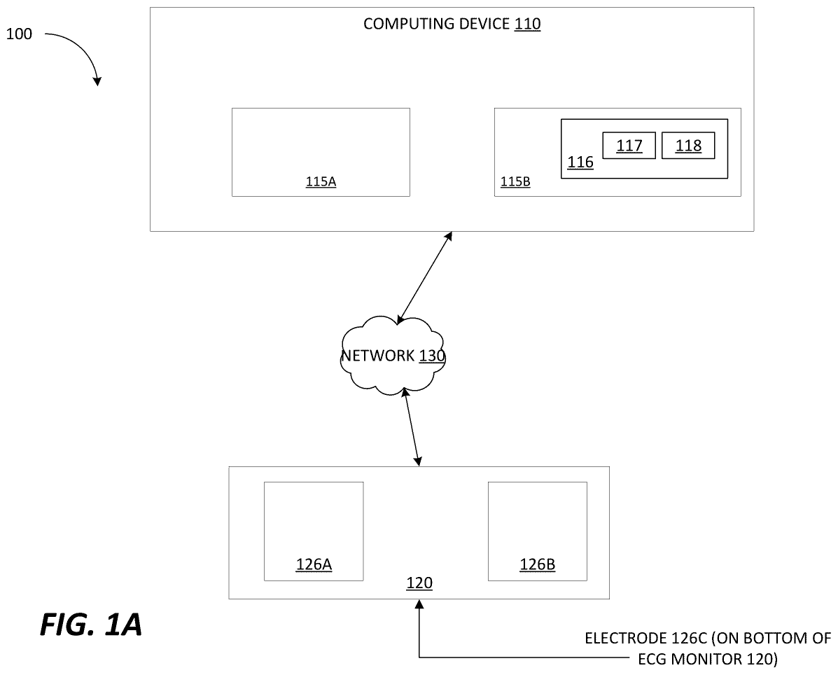

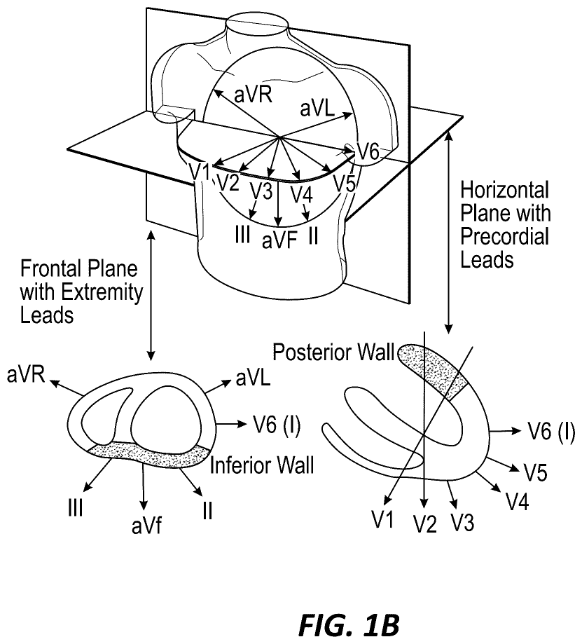

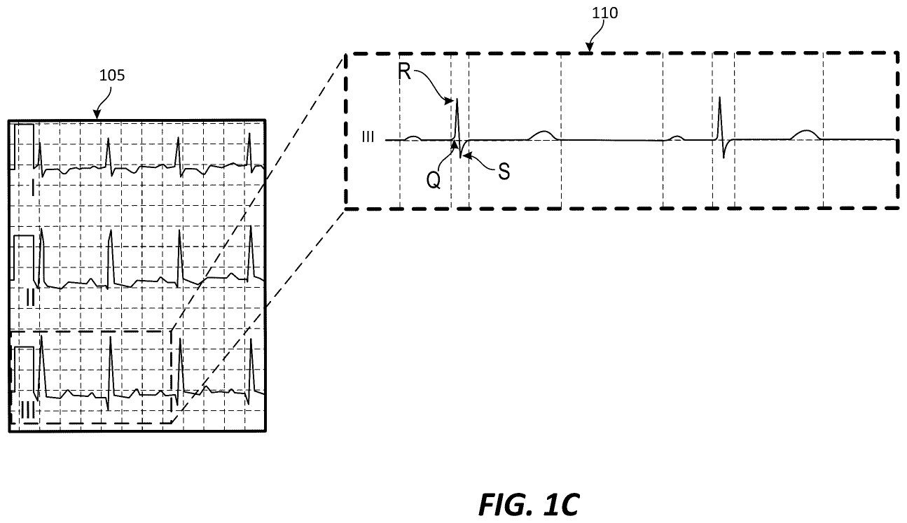

As noted above, ECG waveforms are generated from measuring multiple leads (each lead formed by a different electrode pair), and the ECG waveform obtained from each different electrode pair/lead may be different/unique (e.g., may have different morphologies/amplitudes). This is because although the various leads may analyze the same electrical events, each one may do so from a different angle. FIG. 1C illustrates a view 105 of an ECG waveform detected by each of 3 leads (I, II, and III) when a 3-lead ECG is taken as well as an exploded view 110 of the ECG waveform measured by lead III illustrating the QRS complex. As shown, the amplitudes and morphologies of the ECG waveform taken from leads I-III are all different, with the ECG waveform measured by lead III having the largest amplitude and the ECG waveform measured by lead I having the smallest amplitude.

Numerous applications that run on computing devices (e.g., smart phones, laptop computers etc.) contain and process sensitive personal information. One example is healthcare applications such as the Kardia™ application by AliveCor™ Inc. that displays, analyzes, and manages ECG signals received from an ECG monitor. Healthcare applications contain a user's sensitive health data, but access to them is often-times only protected by the security measures the computing device implements to control access to the computing device itself (e.g., smart phone password). Indeed, many applications do not have their own dedicated password or other security mechanism, so a person who has access to the computing device can access all of the information therein. This can impact a user's comfort with such applications and can also potentially cause legal issues as many governments have laws or are considering laws that require a certain level of security for applications that contain sensitive personal healthcare information (or any type of sensitive personal information). In addition, requiring a password for an application itself could cause a degradation in the user experience, since accessing the application now requires an extra step and an extra password to remember.

Applications are not the only resource that are subject to security/access control concerns. Indeed, many highly secure installations and machines use multi-factor authentication systems utilizing multiple authentication factors to provide heightened security. The factors that a multi-factor authentication system can use may include: what a person knows (e.g., passwords), what a person has (e.g., Verify™ application by Okta™, Google Authentication™, RSA SecurID™), and who a person is (e.g., biometrics such as fingerprints and retina scans). However, passwords and security tokens can be easily stolen. Although biometric authentication is more effective, it is also not completely effective as accidents can destroy finger prints or the eye. It is also possible to use surgically removed body parts to break into this type of biometric security. Similarly to applications, requiring a password for a resource such as a door lock or a machine could cause a degradation in the user experience since accessing the resource now requires an extra step and an extra password to remember.

However, heartbeat characteristics are nearly impossible to mimic. A person's heartbeat is consistent/reliable and is nearly impossible to fake since the various characteristics of a specific person's heartbeat cannot be replicated without the person actually being present. In addition, the various characteristics of a specific person's heartbeat cannot be detected without taking an ECG using an ECG monitor. But using this integrated approach we can increase security without degrading user experience.

The present disclosure addresses the above-noted and other deficiencies by providing systems and methods for performing an ECG search based on a dual ECG and text embedding model. A system may comprise an electrocardiogram (ECG) monitor configured to measure electrical activity of a heart of a user and generate an ECG signal based on the measured electrical activity and computing device associated with a resource. The computing device may be configured to receive a set of ECG signals of a user from the ECG monitor and determine an ECG profile for the user based on the set of ECG signals. In response to receiving a request to access the resource, the computing device may request an ECG to authenticate the user. The computing device may receive an authentication ECG signal from the ECG monitor and compare the authentication ECG signal to the ECG profile of the user to determine whether the authentication ECG signal matches the ECG profile. In response to determining that the subsequent ECG signal matches the ECG profile, the computing device may authenticate the user and grant the user access to the resource.

FIG. 1A shows a system 100 in which embodiments of the present disclosure may be realized. The system 100 may comprise a computing device 110 and an ECG monitor 120. The computing device 110 may be any appropriate computing device, such as a tablet computer, a smartphone, a server computer, a desktop computer, a laptop computer, or a body-worn computing device (e.g., a smart watch or other wearable), for example. In some embodiments, the computing device 110 may comprise a single computing device or may include multiple interconnected computing devices (e.g., multiple servers configured in a cluster). The computing device 110 may include hardware such as processing device 115A (e.g., processors, central processing units (CPUs)), memory 115B (e.g., random access memory (RAM), storage devices (e.g., hard-disk drive (HDD), solid-state drive (SSD), etc.)), a network interface configured to connect with network 130, and other hardware devices (e.g., sound card, video card, etc.). In some embodiments, the memory 115B may be a persistent storage that is capable of storing data. A persistent storage may be a local storage unit or a remote storage unit. Persistent storage may be a magnetic storage unit, optical storage unit, solid state storage unit, electronic storage units (main memory), or similar storage unit. Persistent storage may also be a monolithic/single device or a distributed set of devices. The memory 115B may be configured for long-term storage of data and may retain data between power on/off cycles of the computing device 110. The memory 115B may include a monitoring application 116 which may provide logic and a user interface for receiving, analyzing, visualizing, and managing biometric data (e.g., ECG signals) collected by the ECG monitor 120.

The computing device 110 and the ECG monitor 120 may be coupled to each other (e.g., may be operatively coupled, communicatively coupled, may communicate data/messages with each other) via network 130. Network 130 may be a public network (e.g., the internet), a private network (e.g., a local area network (LAN) or wide area network (WAN)), or a combination thereof. In one embodiment, network 130 may include a wired or a wireless infrastructure, which may be provided by one or more wireless communications systems, such as a Wi-Fi hotspot connected with the network 130 and/or a wireless carrier system that can be implemented using various data processing equipment, communication towers (e.g., cell towers), etc. The network 130 may carry communications (e.g., data, message, packets, frames, etc.) between the computing device 110 and the ECG monitor 120.

When taking an ECG of a person, there are different “standard” configurations for electrode placement that can be used to place electrodes on the person. For example, an electrode placed on the right arm can be referred to as RA. The electrode placed on the left arm can be referred to as LA. The RA and LA electrodes may be placed at the same location on the left and right arms, preferably near the wrist in some embodiments. The leg electrodes can be referred to as RL for the right leg and LL for the left leg. The RL and LL electrodes may be placed on the same location for the left and right legs, preferably near the ankle in some embodiments. Lead I is typically the voltage between the left arm (LA) and right arm (RA), e.g. I=LA−RA. Lead II is typically the voltage between the left leg (LL) and right arm (RA), e.g. II=LL−RA. Lead III is the typically voltage between the left leg (LL) and left arm (LA), e.g. III=LL−LA. Augmented limb leads can also be determined from RA, RL, LL, and LA. The augmented vector right (aVR) lead is equal to RA−(LA+LL)/2 or −(I+II)/2. The augmented vector left (aVL) lead is equal to LA−(RA+LL)/2 or I−II/2. The augmented vector foot (aVF) lead is equal to LL−(RA+LA)/2 or II−I/2. FIG. 1B illustrates a 12 lead set comprising the I, II, III, aVR, aVL, aVF, V1, V2, V3, V4, V5, and V6 leads, all represented on a hexaxial system.

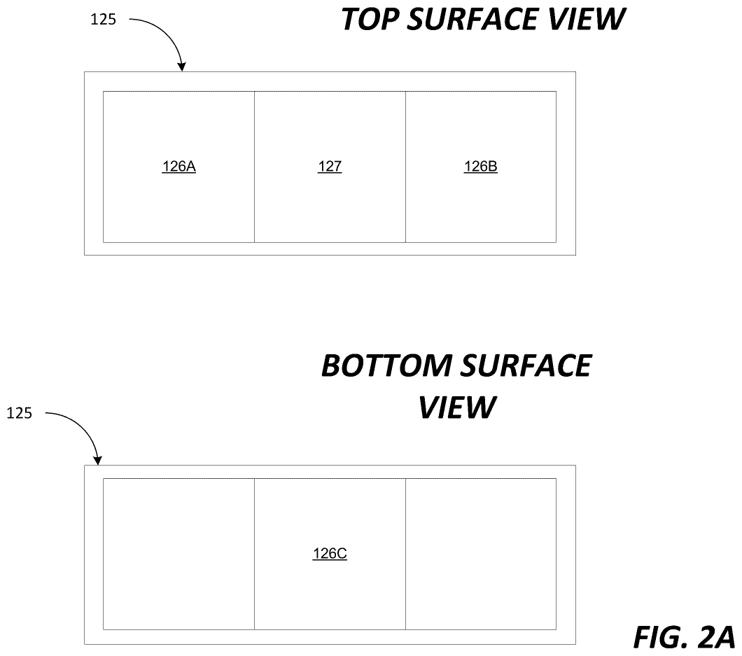

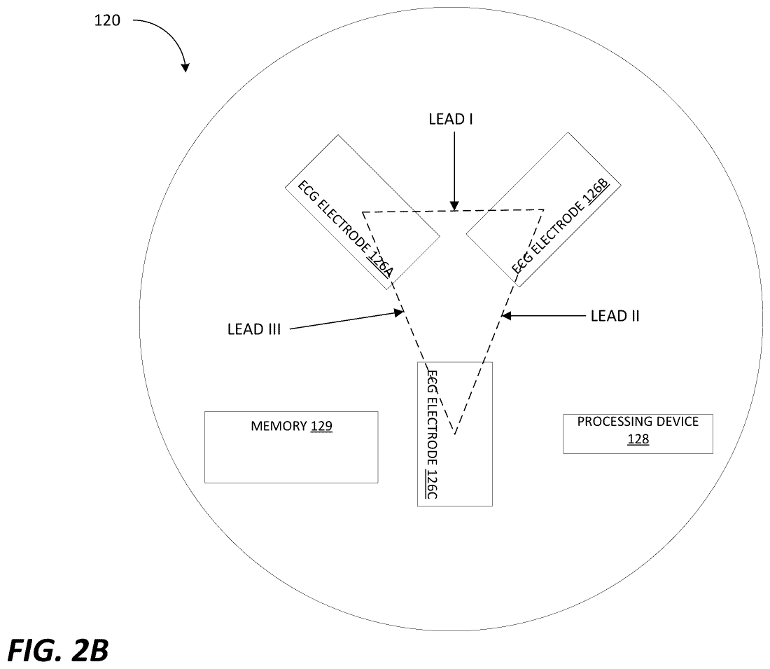

The ECG monitor 120 may comprise any appropriate ECG measuring hardware/software to take an ECG of a patient such as a conventional 10 electrode ECG monitoring device (e.g., a Holter monitor) or a handheld ECG monitor (such as the KardiaMobile® or KardiaMobile® 6L device from AliveCor® Inc., for example) having a reduced form factor/reduced lead set as discussed in further detail with respect to FIGS. 2A-2B. The ECG monitor 120 may further include software/logic corresponding to a machine learning (ML) module for synthesizing one or more leads of the ECG e.g., based on one or more leads measured by the electrodes of the ECG monitor 120 as discussed herein. FIGS. 2A-2B illustrate an example of the ECG monitor 120 of FIG. 1A, which may include a housing 125, where two electrodes 126A and 126B are positioned on a top surface of the housing 125 and a third electrode 126C is positioned on a bottom surface of the housing 125 as shown in FIG. 2A. The electrodes 126 may be insulated from each other via dialectrics 127 or other suitable materials such that they are able to sense and record distinct signals. In some embodiments, the electrodes 126 may be comprised of silver-silver chloride (or some other suitable material) electrodes. In some embodiments, ECG monitor 120 may include an electrode connector (not shown) such as e.g., a female socket on one end or a side allowing one or more external electrodes (not shown) to be connected to the ECG monitor 120 to be used on skin with an adhesive or without an adhesive (e.g., a conductive gel and the electrodes 126).

FIG. 2B illustrates a hardware block diagram of ECG monitor 120, which may include hardware such as processing device 128 (e.g., processors, central processing units (CPUs)), memory 129 (e.g., random access memory (RAM), storage devices (e.g., hard-disk drive (HDD)), solid-state drives (SSD), etc.), and other hardware devices (e.g., analog to digital converter (ADC) etc.). A storage device may comprise a persistent storage that is capable of storing data. A persistent storage may be a local storage unit or a remote storage unit. Persistent storage may be a magnetic storage unit, optical storage unit, solid state storage unit, electronic storage units (main memory), or similar storage unit. Persistent storage may also be a monolithic/single device or a distributed set of devices. In some embodiments, the processing device 128 may comprise a dedicated ECG waveform processing and analysis chip that provides built-in leads off detection. The ECG monitor 120 may include an ADC (not shown) having a high enough sampling frequency for accurately converting the ECG waveforms measured by the set of electrodes 126 into digital signals (e.g., a 24 bit ADC operating at 500 Hz or higher) for processing by the processing device 128.

The electrodes 126 may measure a subset of the leads illustrated in FIG. 1B, such as lead I (e.g., the voltage between the left arm and right arm) contemporaneously with lead II (e.g., the voltage between the left leg and right arm), and lead I contemporaneously with lead V2 or another one of the chest leads such as V5. It should be noted that any other combination of leads is possible. If desired, additional leads can then be algorithmically derived (e.g., by the ECG monitor 120 itself or the computing device 110) from the determined subset of leads. For example, augmented limb leads can also be determined from the values measured by the LA, RA, LL, and RL electrodes. The augmented vector right (aVR) may be equal to RA−(LA+LL)/2 or −(I+II)/2. The augmented vector left (aVL) may be equal to LA−(RA+LL)/2 or I−II/2. The augmented vector foot (aVF) may be equal to LL−(RA+LA)/2 or II−I/2. In some embodiments, the ECG monitor 120 itself or the computing device 110 may utilize a machine learning (ML) model to derive the full 12 lead set from a measured subset of leads. In some embodiments, the ECG monitor 120 may be in the form of a smartphone, or a wearable device such as a smart watch. Although illustrated as a 3 electrode device, ECG monitoring device 120 may include any appropriate number of electrodes forming any appropriate number of leads. In some embodiments, the ECG monitor 120 may be a handheld sensor coupled to the computing device 110 with an intermediate protective case/adapter.

As noted above, ECG waveforms are generated from measuring multiple leads (each lead formed by a different electrode pair), and the ECG waveform obtained from each different electrode pair/lead may be different/unique (e.g., may have different morphologies/amplitudes). This is because although the various leads may analyze the same electrical events, each one may do so from a different angle. FIG. 1C illustrates a view 105 of an ECG waveform detected by each of 3 leads (I, II, and III) when a 3-lead ECG is taken as well as an exploded view 110 of the ECG waveform measured by lead III illustrating the QRS complex. As shown, the amplitudes and morphologies of the ECG waveform taken from leads I-III are all different, with the ECG waveform measured by lead III having the largest amplitude and the ECG waveform measured by lead I having the smallest amplitude.

Referring back to FIG. 1A, the monitoring application 116 may include a biometric authentication module 117 which may provide logic for generating an ECG profile of the user and comparing ECG signals received from the ECG monitor 120 to the ECG profile of the user in order to authenticate the user, as discussed in further detail herein.

In some embodiments, the processing device 115A (executing the biometric authentication module 117) may initiate a registration process. During the registration process, the user may take an ECG using the ECG monitor 120 multiple times through a day (or any appropriate time period) as instructed by e.g., their physician or health care provider and the ECG monitor 120 may transmit the resulting ECG signal from each ECG taken to the computing device 110. The ECG signal may be provided to the monitoring application 116 (and thus the biometric authentication module 117). After a threshold number of ECG signals have been received, the biometric authentication module 117 may generate an ECG profile 118 for the user based on characteristics of the user's ECG signals (which correspond to the characteristics of the user's heartbeat). Stated differently, the characteristics of the user's heartbeat may be indicated by the electrical activity of the user's heart. It should be noted that once the registration process has been initiated, the user can also take all of the threshold number of ECGs at once, to speed up the process of generating the ECG profile 118.

As discussed herein, a typical heartbeat may include several variations of electrical potential, which may be classified into waves, complexes, and segments. The amplitude, shape, and duration of these waves, complexes, and segments, as well as the intervals between certain waves and the ratios between certain waves all represent characteristics of the user's heartbeat that can be used as distinguishing factors and may be referred to hereinafter as ECG metrics. The waves and complexes may include e.g., P, Q, R and S waves, a QRS complex, a T wave, and sometimes a U wave as is known in the art. For example, the shape and duration of the P wave can be related to the size of the user's atrium (e.g., indicating atrial enlargement) and can be a first source of heartbeat characteristics unique to a user. The amplitude, shape, and duration of each of the Q, R and S waves (ECG metrics) can vary in different individuals, and in particular can vary significantly for users having cardiac diseases or cardiac irregularities. In another example, a Q wave that is greater than ⅓ of the height of the R wave, or greater than 40 ms in duration can be indicative of a myocardial infarction and provide a unique characteristic of the user's heart. Similarly, other healthy ratios of Q and R waves can be used to distinguish different users' heartbeats. Other characteristics that can be used as distinguishing factors include the ST segment, the ST-T segment, the PR interval (distance from onset of the P wave to onset of the QRS complex), RR interval (distance between R waves), PP interval (distance between P waves), the QT duration, the TP interval, and the heart rate variability (HRV), among other examples. As can be seen, there are a variety of different characteristics of a user's heartbeat that can be captured by the ECG metrics of an ECG signal and can be used to define the ECG profile 118 (i.e., set of heartbeat characteristics) that is unique to the user.

As the biometric authentication module 117 continues to receive ECG signals over time, it may continue to learn the values of each of the above ECG metrics that define the ECG profile 118 of the user. In some embodiments, the ECG profile 118 of the user may be based on a subset of the above ECG metrics. The biometric authentication module 117 may require a threshold number of ECG signals before the ECG profile 118 is considered complete. The threshold number of ECG signals may be tuneable by e.g., the developer of the monitoring application 116 or even the user in some scenarios based on any of a number of factors.

In some embodiments, the biometric authentication module 117 may comprise an ML model that may learn/generate the ECG metrics that will ultimately define the user's ECG profile 118 by being trained based on the ECG signals received over time. ML models are well suited for continuous monitoring of one or multiple criteria to identify anomalies or trends, big and small, in input data as compared to training examples used to train the model. The ML models described herein may be trained on ECG data from a population of users, and/or trained on other training examples to suit the design needs for the model. Machine learning models that may be used with embodiments described herein include by way of example and not limitation: Bayes, Markov, Gausian processes, clustering algorithms, generative models, kernel and neural network algorithms. Some embodiments utilize a machine learning model based on a trained neural network (e.g., a trained recurrent neural network (RNN) or a trained convolution neural network (CNN)).

For example, an ML model may comprise a trained CNN ML model that takes input data (e.g., ECG data) into convolutional layers (aka hidden layers), and applies a series of trained weights or filters to the input data in each of the convolutional layers. The output of the first convolutional layer is an activation map, which is the input to the second convolution layer, to which a trained weight or filter (not shown) is applied, where the output of the subsequent convolutional layers results in activation maps that represent more and more complex features of the input data to the first layer. After each convolutional layer a non-linear layer (not shown) is applied to introduce non-linearity into the problem, which nonlinear layers may include an activation function such as tanh, sigmoid or ReLU. In some cases, a pooling layer (not shown) may be applied after the nonlinear layers, also referred to as a downsampling layer, which basically takes a filter and stride of the same length and applies it to the input, and outputs the maximum number in every sub-region the filter convolves around. Other options for pooling are average pooling and L2-normalization pooling. The pooling layer reduces the spatial dimension of the input volume reducing computational costs and to control overfitting. The final layer(s) of the network is a fully connected layer, which takes the output of the last convolutional layer and outputs an n-dimensional output vector representing the quantity to be predicted. This may result in a predictive output. The trained weights may be different for each of the convolutional layers.

To achieve real-world prediction/detection, a neural network needs to be trained on known data inputs or training examples, thereby resulting in a trained neural network. To train a neural network, many different training examples (e.g., ECG signals of a user at different times) are input into the model. A skilled artisan in neural networks will fully understand the description above provides a somewhat simplistic view of neural networks to provide some context for the present discussion and will fully appreciate the application of any neural network alone or in combination with other neural networks or other entirely different machine learning models will be equally applicable and within the scope of some embodiments described herein.

Once the ECG profile 118 of the user is generated, the biometric authentication module 117 may act as a “gate keeper” of the monitoring application 116. More specifically, when the user wishes to access the monitoring application 116 (e.g., attempts to login), the biometric authentication module 117 may display a notice on the display of computing device 110 indicating that the user must be authenticated via ECG. The user may subsequently take an ECG using the ECG monitor 120, which may transmit the resulting ECG signal to the computing device 110 (and particularly, the biometric authentication module 117). The ECG signal generated by the ECG monitor 120 in response to an authentication request may be referred to herein as an authentication ECG signal. In some embodiments, the biometric authentication module 117 may deny access to the monitoring application 116 if an authentication ECG signal is not received within a threshold amount of time.

Upon receiving the authentication ECG signal, the biometric authentication module 117 may compare the authentication ECG signal to the ECG profile 118 of the user to determine if they match (i.e., determine if the person who took the ECG using the ECG monitor 120 is indeed the user). If there is a match between the authentication ECG signal and the ECG profile 118, the biometric authentication module 117 may authenticate the user. More specifically, the biometric authentication module 117 may compare the ECG metrics that define the ECG profile 118 to the ECG metrics of the authentication ECG signal. The biometric authentication module 117 may determine whether each ECG metric of the authentication ECG signal is within a tolerance threshold of the corresponding ECG metric of the ECG profile 118. The tolerance threshold for each ECG metric may be preset and may be different for each ECG metric. In addition, the tolerance threshold for each ECG metric may be tuneable to decrease or increase the level of accuracy required for authentication. In some embodiments, each of the ECG metrics of the authentication signal must be within the tolerance threshold of the corresponding ECG metric of the ECG profile 118 in order for the biometric authentication module 117 to determine that they match. In other embodiments, only a threshold number of the ECG metrics of the authentication signal must be within the tolerance threshold of the corresponding ECG metric of the ECG profile 118 in order for the biometric authentication module 117 to determine that they match. The threshold number of the ECG metrics of the authentication signal that must be within the tolerance threshold of the corresponding ECG metric of the ECG profile 118 may also be a parameter that is tuneable by e.g., the developer of the monitoring application 116 or even the user in some scenarios based on any of a number of factors. Once the biometric authentication module 117 determines that the authentication ECG signal matches the ECG profile 118, it may grant the user access to the monitoring application 116. It should be noted that although illustrated with the ECG profile 118 being stored on the computing device 110 itself, this is not a limitation and the ECG profile 118 may be stored using a cloud storage system or any other appropriate storage system.

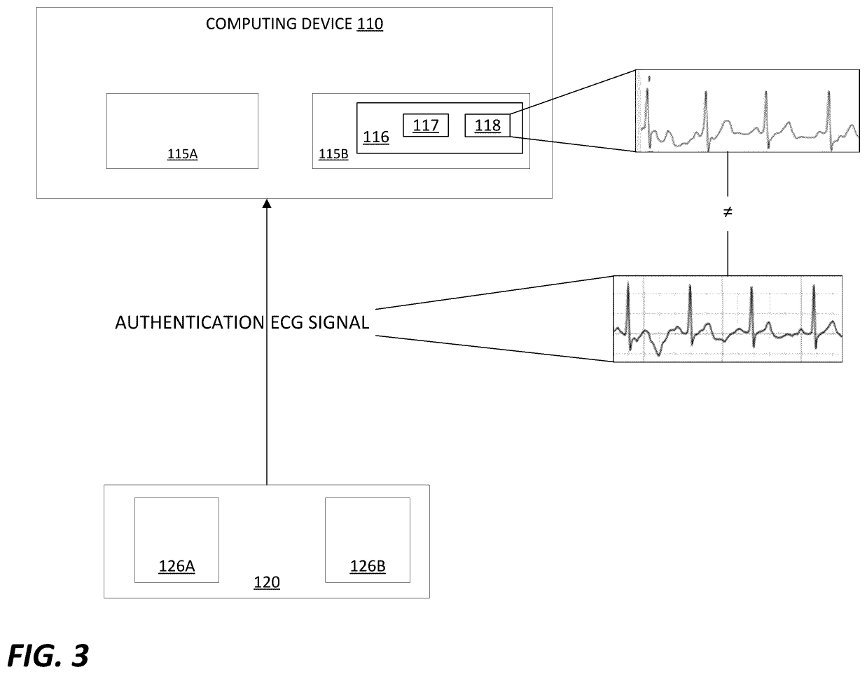

FIG. 3 illustrates an example of the ECG profile 118 and authentication ECG signal comparison process. As shown in FIG. 3, the ECG profile 118 may have ECG metrics of: a PR interval of 0.12 seconds, a P wave duration of 0.7 seconds, a P wave distance of 1.7 mm, and an R wave amplitude of 14 mm. The ECG monitor 120 may generate an authentication ECG signal having ECG metrics of: a PR interval of 0.18 seconds, a P wave duration of 0.11 seconds, a P wave distance of 2.3 mm, and an R wave amplitude of 18 mm. The PR interval tolerance threshold may be 0.03 seconds, the P wave duration tolerance threshold may be 0.2 seconds, the P wave distance tolerance threshold may be 0.2 mm, and the R wave amplitude tolerance threshold may be 5 mm. Upon receiving the authentication ECG signal, the biometric authentication module 117 may compare the authentication ECG signal to the ECG profile 118 as discussed herein and determine they are not a match. More specifically, only the R wave amplitude of the authentication ECG signal is within the tolerance threshold of the corresponding ECG metric, while the other ECG metrics are not. Thus, the biometric authentication module 117 may determine that the authentication ECG signal and the ECG profile 118 are not a match and may deny access to the monitoring application 116.

Referring back to FIG. 1A, in some embodiments, instead of initiating a registration process, the biometric authentication module 117 can utilize ECG signals that have been stored in the memory 115B from previous uses of the monitoring application 116 and the ECG monitor 120. In these embodiments, the biometric authentication module 117 may generate the ECG profile 118 as discussed hereinabove using the stored previous ECG signals and perform authentication as also discussed hereinabove.

In some embodiments, the ECG-based authentication described herein may be implemented as one factor in a multi-factor authentication system. Thus, when the user is logging into the monitoring application 116, the ECG-based authentication may be a first, intermediate, or final authentication factor. In an example where the ECG-based authentication is an intermediate authentication factor and an initial authentication factor is a username and password challenge, the computing device 110 (executing the biometric authentication module 117) may request the authentication ECG signal from the user as discussed hereinabove in response to the user providing the correct username and password (the first authentication factor). If the ECG-based authentication is successful, then the computing device 110 may implement the final authentication factor. In this way, every time the user logs into the monitoring application 116, they will be required to take their ECG and the biometric authentication module 117 will receive the authentication ECG signal and compare it to the user's ECG profile 118. If authentication ECG signal matches the ECG profile 118 (and the other authentication factors as applicable have been satisfied), the biometric authentication module 117 will authenticate the user and grant the user access to the monitoring application 116.

In some embodiments, the computing device 110 may utilize standard multi-factor authentication to secure a particular resource using any appropriate factors (e.g., password, fingerprint scan, RSA token) until the ECG profile 118 for the user has been created. Upon determining that the ECG profile 118 has been created, the computing device 110 may switch to using multi-factor authentication with one factor being the ECG biometric authentication as described herein. Stated differently, the computing device 110 may swap out one of the currently implemented factors in its multi-factor authentication system for ECG biometric authentication. The ECG biometric authentication may be the primary factor, the secondary factor, or the third factor (if three factor authentication is being used).

In some embodiments, anytime the ECG monitor 120 sends an ECG signal or the ECG authentication signal to the computing device 110, it may encrypt the ECG signal before doing so. In this way, the ECG signal can be protected from any form of cyber-intrusion. Any appropriate encryption algorithm may be used including asymmetric and symmetric encryption algorithms. The biometric authentication module 117 may receive the ECG signal and decrypt the encrypted ECG signal and proceed to compare it to the ECG profile 118 (if the received signal is the authentication ECG signal).

In some embodiments, upon generation of the ECG profile 118, the biometric authentication module 117 may create a hash of the ECG profile 118. Thus, when it receives an authentication ECG signal from the ECG monitor 120, it may decrypt it as necessary and generate a hash of the received authentication ECG signal. The biometric authentication module 117 may then compare the hashed authentication ECG signal to the hashed ECG profile 118 to determine whether the hashes match. If the hashes match, the biometric authentication module 117 may also ensure the integrity of the authentication ECG signal file (i.e., that it has not been tampered with).

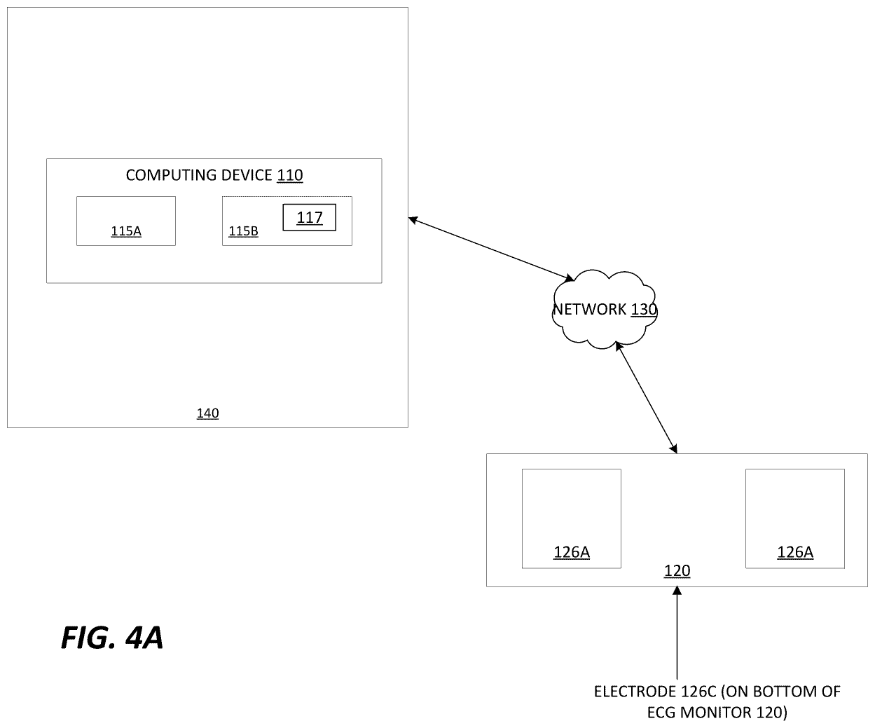

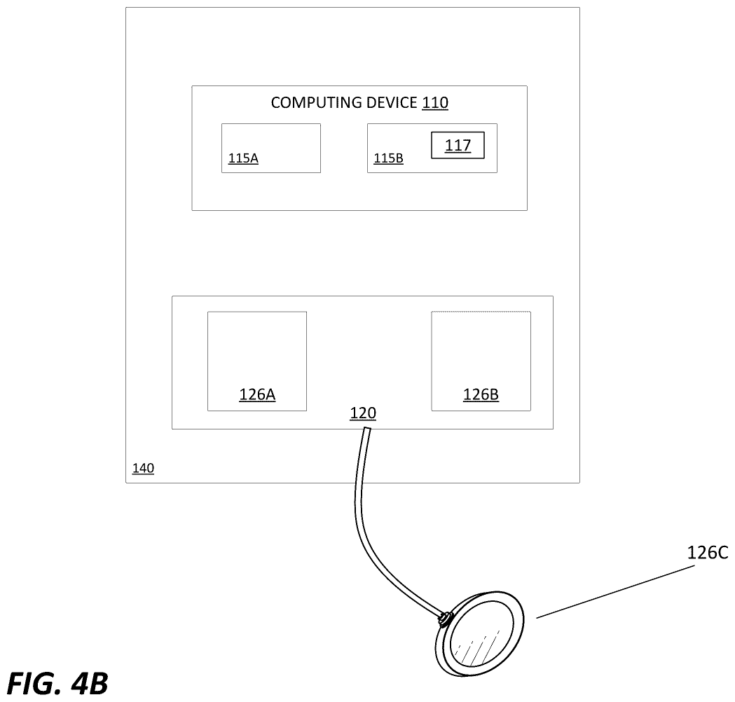

The techniques described herein are not limited to controlling access to an application and indeed, the monitoring application 116 is just one example of a resource for which access can be controlled by the techniques described herein. In some embodiments, the biometric authentication module 117 may be used to control access to other resources. FIG. 4A illustrates an example system in which the biometric authentication module 117 may be used to control access to a resource 140 that is associated with the computing device 110. The resource 140 may be a machine (e.g., a bottling plant, a welding station, etc.), may be a secure facility (e.g., an entry door to the secure facility or a door lock thereof), or other resource for which access needs to be authenticated before the resource can be made available. The computing device 110 may be coupled to the resource 140 or may be implemented using on-board hardware of the resource 140 (e.g., may be an on-board computing device of a machine).

In some embodiments, each user who may potentially access the resource 140 carries a respective ECG monitor, and the ECG monitor 120 shown in FIG. 4A may be an ECG monitor that one particular user who may potentially access the resource 140 carries with them. The biometric authentication module 117 may perform a registration process as described herein to generate and store an ECG profile for each of a number of different users. The ECG profile for each user may be identified by a device ID of that user's respective ECG monitor, a user ID of that user, or any other appropriate identifying information. The identifying information may also be included with each ECG signal transmitted to the biometric authentication module 117 by each user's ECG monitor so that the biometric authentication module 117 knows which user a received ECG signal corresponds to and thus which ECG profile the received ECG signal contributes to.

As shown in FIG. 4B, in some embodiments the ECG monitor 120 may also be coupled to the resource 140 along with the computing device 110, such that each user who may potentially access the resource 140 must use the same ECG monitor 120 to authenticate and access the resource. In such embodiments, the computing device 110 may provide any other appropriate techniques to identify each individual user and their ECG profile/ECG signals during the registration process. Such techniques may include e.g., usernames and/or passwords, finger prints, and retina scanning. For example, if the ECG-based authentication is one factor in a multi-factor authentication system, during the registration process the first factor can be used to identify the user, and the biometric authentication module 117 may then know which user is using the ECG monitor 120 to provide an ECG signal so that the biometric authentication module 117 can learn their ECG profile. As the biometric authentication module 117 learns the ECG profile of each user, ECG-based authentication as discussed hereinabove can be used as an intermediate or final factor for authentication of that user. As shown in FIG. 4B, because the ECG monitor 120 is integrated with the resource 140, the electrode 126C may be implemented as a separate electrode that is connected via wire to the housing 125 of the ECG monitor 120.

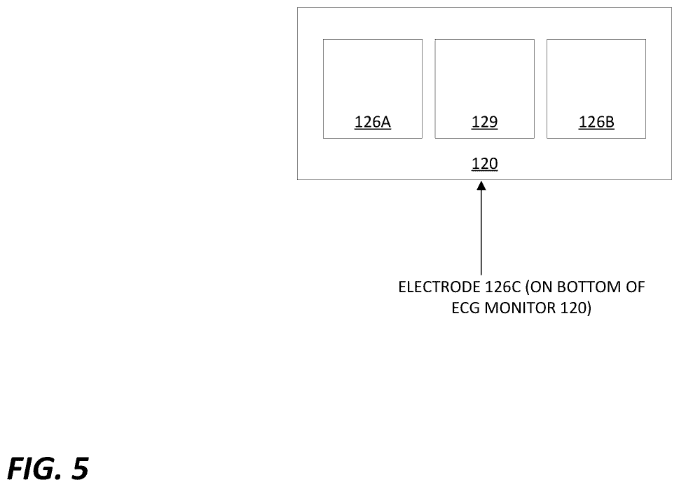

In some embodiments, the ECG monitor 120 may include a fingerprint reader 131 so that it can function as a combined fingerprint and ECG reader. In the example shown in FIG. 5, the fingerprint reader 131 may be integrated into the ECG monitor 120 between the electrodes 126A and 126B. In this way, the user may position their left index finger over the electrode 126A and the fingerprint reader 131 simultaneously and position their right index finger over the electrode 126B and the fingerprint reader 131 simultaneously so that authentications for the user's fingerprint as well as ECG can be done at the same time.

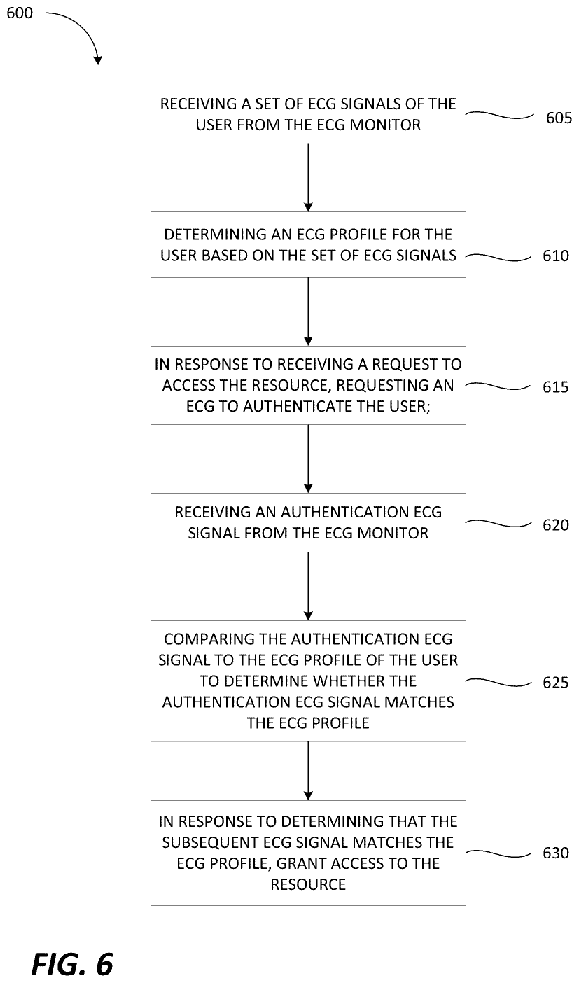

FIG. 6 is a flow diagram of a method 600 for performing ECG-based biometric authentication, in accordance with some embodiments of the present disclosure. Method 600 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In some embodiments, the method 600 may be performed by a computing device (e.g., computing device 110 illustrated in FIG. 1A).

Referring simultaneously to FIG. 1A, the method 600 begins at block 605 where the biometric authentication module 117 may receive a set of ECG signals. In some embodiments the processing device 115A (executing the biometric authentication module 117) may initiate a registration process. During the registration process, the user may take an ECG using the ECG monitor 120 multiple times through a day (or any appropriate time period) as instructed by e.g., their physician or health care provider and the ECG monitor 120 may transmit the resulting ECG signal from each ECG taken to the computing device 110. The ECG signal may be provided to the monitoring application 116 (and thus the biometric authentication module 117). After a threshold number of ECG signals have been received, at block 610 the biometric authentication module 117 may generate an ECG profile 118 for the user based on characteristics of the user's ECG signals (which correspond to the characteristics of the user's heartbeat). Stated differently, the characteristics of the user's heartbeat may be indicated by the electrical activity of the user's heart. It should be noted that once the registration process has been initiated, the user can also take all of the threshold number of ECGs at once, to speed up the process of generating the ECG profile 118.

As the biometric authentication module 117 continues to receive ECG signals over time, it may continue to learn the values of each of the above ECG metrics that define the ECG profile 118 of the user. In some embodiments, the ECG profile 118 of the user may be based on a subset of the above ECG metrics. The biometric authentication module 117 may require a threshold number of ECG signals before the ECG profile 118 is considered complete. The threshold number of ECG signals may be tuneable by e.g., the developer of the monitoring application 116 or even the user in some scenarios based on any of a number of factors.

Once the ECG profile 118 of the user is generated, the biometric authentication module 117 may act as a “gate keeper” of the monitoring application 116. More specifically, when the user wishes to access the monitoring application 116 (e.g., attempts to login), at block 615 the biometric authentication module 117 may display a notice on the display of computing device 110 indicating that the user must be authenticated via ECG. The user may subsequently take an ECG using the ECG monitor 120, which may at block 620 transmit the resulting ECG signal to the computing device 110 (and particularly, the biometric authentication module 117). The ECG signal generated by the ECG monitor 120 in response to an authentication request may be referred to herein as an authentication ECG signal. In some embodiments, the biometric authentication module 117 may deny access to the monitoring application 116 if an authentication ECG signal is not received within a threshold amount of time.

Upon receiving the authentication ECG signal, at block 625 the biometric authentication module 117 may compare the authentication ECG signal to the ECG profile 118 of the user to determine if they match (i.e., determine if the person who took the ECG using the ECG monitor 120 is indeed the user). More specifically, the biometric authentication module 117 may compare the ECG metrics that define the ECG profile 118 to the ECG metrics of the authentication ECG signal. The biometric authentication module 117 may determine whether each ECG metric of the authentication ECG signal is within a tolerance threshold of the corresponding ECG metric of the ECG profile 118. At block 630, if there is a match between the authentication ECG signal and the ECG profile 118, the biometric authentication module 117 may authenticate the user and grant the user access to the monitoring application (or other resource).

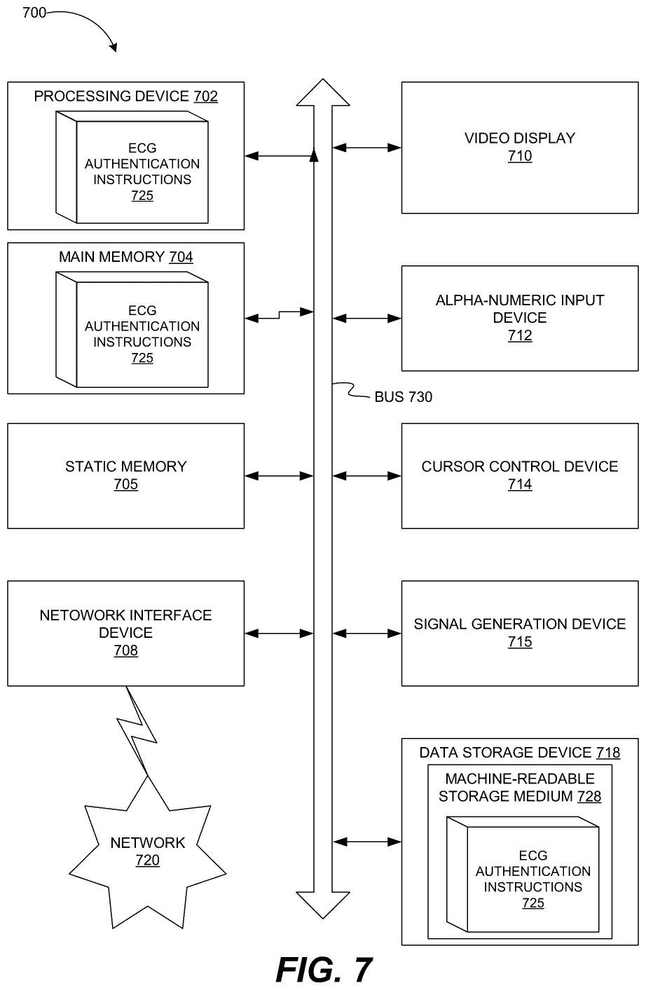

FIG. 7 illustrates a diagrammatic representation of a machine in the example form of a computer system 700 within which a set of instructions, for causing the machine to perform any one or more of the methodologies discussed herein for performing an ECG search.

In alternative embodiments, the machine may be connected (e.g., networked) to other machines in a local area network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, a switch or bridge, a hub, an access point, a network access control device, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. In one embodiment, computer system 700 may be representative of a server.

The exemplary computer system 700 includes a processing device 702, a main memory 704 (e.g., read-only memory (ROM), flash memory, dynamic random access memory (DRAM), a static memory 706 (e.g., flash memory, static random access memory (SRAM), etc.), and a data storage device 718, which communicate with each other via a bus 730. Any of the signals provided over various buses described herein may be time multiplexed with other signals and provided over one or more common buses. Additionally, the interconnection between circuit components or blocks may be shown as buses or as single signal lines. Each of the buses may alternatively be one or more single signal lines and each of the single signal lines may alternatively be buses.

Computing device 700 may further include a network interface device 708 which may communicate with a network 720. The computing device 700 also may include a video display unit 710 (e.g., a liquid crystal display (LCD) or a cathode ray tube (CRT)), an alphanumeric input device 712 (e.g., a keyboard), a cursor control device 714 (e.g., a mouse) and an acoustic signal generation device 716 (e.g., a speaker). In one embodiment, video display unit 710, alphanumeric input device 712, and cursor control device 714 may be combined into a single component or device (e.g., an LCD touch screen).

Processing device 702 represents one or more general-purpose processing devices such as a microprocessor, central processing unit, or the like. More particularly, the processing device may be complex instruction set computing (CISC) microprocessor, reduced instruction set computer (RISC) microprocessor, very long instruction word (VLIW) microprocessor, or processor implementing other instruction sets, or processors implementing a combination of instruction sets. Processing device 702 may also be one or more special-purpose processing devices such as an application specific integrated circuit (ASIC), a field programmable gate array (FPGA), a digital signal processor (DSP), network processor, or the like. The processing device 702 is configured to execute ECG authentication instructions 725, for performing the operations and steps discussed herein.

The data storage device 715 may include a machine-readable storage medium 728, on which is stored one or more sets of ECG authentication instructions 725 (e.g., software) embodying any one or more of the methodologies of functions described herein. The ECG authentication instructions 725 may also reside, completely or at least partially, within the main memory 704 or within the processing device 702 during execution thereof by the computer system 700; the main memory 704 and the processing device 702 also constituting machine-readable storage media. The ECG authentication instructions 725 may further be transmitted or received over a network 720 via the network interface device 708.

While the machine-readable storage medium 728 is shown in an exemplary embodiment to be a single medium, the term “machine-readable storage medium” should be taken to include a single medium or multiple media (e.g., a centralized or distributed database, or associated caches and servers) that store the one or more sets of instructions. A machine-readable medium includes any mechanism for storing information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read-only memory (ROM); random-access memory (RAM); erasable programmable memory (e.g., EPROM and EEPROM); flash memory; or another type of medium suitable for storing electronic instructions.

The preceding description sets forth numerous specific details such as examples of specific systems, components, methods, and so forth, in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular embodiments may vary from these exemplary details and still be contemplated to be within the scope of the present disclosure.

Additionally, some embodiments may be practiced in distributed computing environments where the machine-readable medium is stored on and or executed by more than one computer system. In addition, the information transferred between computer systems may either be pulled or pushed across the communication medium connecting the computer systems.

Embodiments of the claimed subject matter include, but are not limited to, various operations described herein. These operations may be performed by hardware components, software, firmware, or a combination thereof.

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent or alternating manner.

The above description of illustrated implementations of the invention, including what is described in the Abstract, is not intended to be exhaustive or to limit the invention to the precise forms disclosed. While specific implementations of, and examples for, the invention are described herein for illustrative purposes, various equivalent modifications are possible within the scope of the invention, as those skilled in the relevant art will recognize. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Furthermore, the terms “first,” “second,” “third,” “fourth,” etc. as used herein are meant as labels to distinguish among different elements and may not necessarily have an ordinal meaning according to their numerical designation.

It will be appreciated that variants of the above-disclosed and other features and functions, or alternatives thereof, may be combined into may other different systems or applications. Various presently unforeseen or unanticipated alternatives, modifications, variations, or improvements therein may be subsequently made by those skilled in the art which are also intended to be encompassed by the following claims. The claims may encompass embodiments in hardware, software, or a combination thereof.

What is claimed is:

1. A system comprising:
an electrocardiogram (ECG) monitor configured to measure electrical activity of a heart of a user and generate an ECG signal based on the measured electrical activity; and
a computing device associated with a resource, the computing device configured to:
receive a set of ECG signals of the user from the ECG monitor;
determine an ECG profile for the user based on the set of ECG signals;
in response to receiving a request to access the resource, request an authentication ECG signal to authenticate the user;
receive the authentication ECG signal from the ECG monitor;
compare the authentication ECG signal to the ECG profile of the user to determine whether the authentication ECG signal matches the ECG profile;
in response to determining that the authentication ECG signal matches the ECG profile, authenticate the user; and
grant the user access to the resource.

2. The system of claim 1, wherein the ECG profile and the authentication ECG signal each comprise ECG metrics representing a set of heartbeat characteristics, the ECG metrics including one or more of an amplitude, a shape, and a duration for each of: a P wave, a Q wave, an R wave, an S wave, a QRS complex, a T wave, an ST segment, an ST-T segment, a PR interval, an RR interval, a PP interval, and a QT duration.

3. The system of claim 2, wherein to compare the authentication ECG signal to the ECG profile of the user, the computing device is to:
compare the ECG metrics of the ECG profile to the ECG metrics of the authentication signal; and
determine that the authentication ECG signal matches the ECG profile if a threshold number of the ECG metrics of the ECG profile are each within a tolerance threshold of their corresponding ECG metrics of the authentication ECG signal.

4. The system of claim 1, wherein the computing device is further to:
generate a hash of the ECG profile;
generate a hash of the authentication signal; and
determine that the authentication ECG signal has not been tampered with by comparing the hash of the ECG profile to the hash of the authentication signal.

5. The system of claim 1, wherein the computing device implements a multi-factor authentication scheme and is further configured to:
in response to receiving the request to access the resource, implement a first authentication factor; and
in response to the user being authenticated by the first authentication factor, request the authentication ECG to authenticate the user.

6. The system of claim 5, wherein the computing device is further to:
in response to determining that the subsequent ECG signal matches the ECG profile, implement a final authentication factor, wherein the computing device grants the user access to the resource in response to authenticating the user by the final authentication factor.

7. The system of claim 1, wherein the resource comprises a biometric monitoring application that is executed on the computing device.

8. The system of claim 1, wherein the resource comprises a machine that is operatively coupled to the computing device.

9. The system of claim 1, wherein the resource comprises a door that is operatively coupled to the computing device.

10. The system of claim 9, and wherein to grant the user access to the door, the computing device is to disarm a lock of the door.

11. A method comprising:
receiving, by a computing device associated with a resource, a set of electrocardiogram (ECG) signals of a user from an ECG monitor;
determining an ECG profile for the user based on the set of ECG signals;
in response to receiving a request to access the resource, requesting an authentication ECG signal to authenticate the user;
receiving the authentication ECG signal from the ECG monitor;
comparing the authentication ECG signal to the ECG profile of the user to determine whether the authentication ECG signal matches the ECG profile;
in response to determining that the authentication ECG signal matches the ECG profile, authenticating the user; and
granting the user access to the resource.

12. The method of claim 11, wherein the ECG profile and the authentication ECG signal each comprise ECG metrics representing a set of heartbeat characteristics, the ECG metrics including one or more of an amplitude, a shape, and a duration for each of: a P wave, a Q wave, an R wave, an S wave, a QRS complex, a T wave, an ST segment, an ST-T segment, a PR interval, an RR interval, a PP interval, and a QT duration.

13. The method of claim 12, wherein comparing the authentication ECG signal to the ECG profile of the user comprises:
comparing the ECG metrics of the ECG profile to the ECG metrics of the authentication signal; and
determining that the authentication ECG signal matches the ECG profile if a threshold number of the ECG metrics of the ECG profile are each within a tolerance threshold of their corresponding ECG metrics of the authentication ECG signal.

14. The method of claim 11, further comprising:
generating a hash of the ECG profile;
generating a hash of the authentication signal; and
determining that the authentication ECG signal has not been tampered with by comparing the hash of the ECG profile to the hash of the authentication signal.

15. The method of claim 11, wherein the computing device implements a multi-factor authentication scheme and the method further comprises:
in response to receiving the request to access the resource, implementing a first authentication factor; and
in response to the user being authenticated by the first authentication factor, requesting the authentication ECG to authenticate the user.

16. The method of claim 15, further comprising:
in response to determining that the subsequent ECG signal matches the ECG profile, implementing a final authentication factor; and
granting the user access to the resource in response to the user being authenticated by the final authentication factor.

17. The method of claim 11, wherein the resource comprises a biometric monitoring application that is executed on the computing device.

18. The method of claim 11, wherein the resource comprises a machine that is operatively coupled to the computing device.

19. The method of claim 11, wherein the resource comprises a door that is operatively coupled to the computing device.

20. The method of claim 19, wherein granting the user access to the door comprises disarming a lock of the door.
